(19) United States
(12) Patent Application Publication
(10) Pub. No.: US 2023/0404454 A1
(43) Pub. Date: Dec. 21, 2023

(54) SUITABILITY STATE DECISION APPARATUS, SUITABILITY STATE DECISION METHOD, AND NON-TRANSOITPORY COMPUTER-READBLE MEDIUM

(71) Applicant: NEC Corporation, Tokyo (JP)

(72) Inventors: Keiju Nakamura, Tokyo (JP); Hiroaki Kiso, Tokyo (JP)

(73) Assignee: NEC Corporation, Tokyo (JP)

(21) Appl. No.: 18/208,079

(22) Filed: Jun. 9, 2023

(30) Foreign Application Priority Data
Jun. 17, 2022 (JP) 2022-098361

(51) Int. Cl. A61B 5/16; G06F 3/16; G06V 40/16

(52) U.S. Cl. CPC A61B 5/165; G06V 40/174; G06F 3/16

(57) ABSTRACT

For supporting a decision whether a medicine is suitable for a target, a suitability state decision apparatus includes an acquisition unit and a decision unit. The acquisition unit acquires a first image being an image when a target takes a medicine. The decision unit generates, by using the first image, suitability state information indicating a suitability state of the target relating to the medicine. The suitability state information includes, for example, at least one of information indicating whether the target feels comfortable to take the medicine and information indicating whether the target feels reluctant to take the medicine.

This application is based on Japanese patent application No. 2022-98361, the content of which is incorporated hereinto by reference.

### BACKGROUND

### Technical Field

The present invention relates to a suitability state decision apparatus, a suitability state decision method, and a non-transitory computer-readable medium staring a program thereof.

### Related Art

It is important to have a target take a medicine. For example, Japanese Patent Application Publication No. 2020-058622 describes that a medication management system for performing medication assistance and medication management acquires image data, also reads specific medication information described in a medication information provision document, and records, as an evidence, the information acquired from the image data and the specific medication information read by an information reading unit in association with each other. Further, Japanese Patent Application Publication No. 2020-058622 also describes that the medication management system records an auxiliary operation performed by a carer as moving image data.

Note that, Japanese Patent Application Publication No. 2001-043345 describes that an image of an expression of a patient during medication is captured, and a content of the medication and a dosage are controlled based on an evaluation result of the expression.

### SUMMARY

In order to have a target take a medicine, it is preferable to decide whether the medicine is suitable for the target. However, none of the documents described above suggest support for a decision whether a medicine is suitable for a target.

One example of an object of the present invention is, in view of the problem described above, to provide a suitability state decision apparatus, a suitability state decision method, and a program that can support a decision whether a medicine is suitable for a target.

In one embodiment, there is provided a suitability state decision apparatus including:
an acquisition unit that acquires a first image being an image when a target takes a medicine; and
a decision unit that generates, by using the first image, suitability state information indicating a suitability state of the target relating to the medicine.

In another embodiment, there is provided a suitability state decision method including,
by a computer:
acquiring a first image being an image when a target takes a medicine; and
generating, by using the first image, suitability state information indicating a suitability state of the target relating to the medicine.

In still another embodiment, there is provided a non-transitory computer-readable medium storing a program for causing a computer to perform operations, the operations comprising:
acquiring a first image being an image when a target takes a medicine; and
generating, by using the first image, suitability state information indicating a suitability state of the target relating to the medicine.

One aspect of the present invention can provide a suitability state decision apparatus, a suitability state decision method, and a program that can support a decision whether a medicine is suitable for a target.

### BRIEF DESCRIPTION OF THE DRAWINGS

The above and other objects, advantages and features of the present invention will be more apparent from the following description of certain preferred example embodiments taken in conjunction with the accompanying drawings, in which:

FIG. 1 is a diagram illustrating an overview of a suitability state decision apparatus according to a first example embodiment;

FIG. 2 is diagram illustrating a usage environment of the suitability state decision apparatus;

FIG. 3 is a diagram illustrating one example of a functional configuration of the suitability state decision apparatus;

FIG. 4 is a diagram illustrating one example of a data configuration of a history storage unit;

FIG. 5 is a diagram illustrating a hardware configuration example of the suitability state decision apparatus;

FIG. 6 is a flowchart illustrating one example of processing performed by the suitability state decision apparatus and a terminal;

FIG. 7 is a flowchart illustrating a first example of step S40 in FIG. 6;

FIG. 8 is a flowchart illustrating a second example of step S40 in FIG. 6;

FIG. 9 is a flowchart illustrating a first modification example of FIG. 6;

FIG. 10 is a flowchart illustrating a second modification example of FIG. 6;

FIG. 11 is a diagram illustrating one example of a functional configuration of a suitability state decision apparatus according to a second example embodiment; and

FIG. 12 is a diagram illustrating one example of a functional configuration of a suitability state decision apparatus according to a third example embodiment.

### DETAILED DESCRIPTION

The invention will be now described herein with reference to illustrative example embodiments. Those skilled in the art will recognize that many alternative example embodiments can be accomplished using the teachings of the present invention and that the invention is not limited to the example embodiments illustrated for explanatory purposes.

Hereinafter, example embodiments of the present invention will be described with reference to the drawings. Note that, in all of the drawings, a similar component has a similar reference sign, and description thereof will be appropriately omitted.

### First Example Embodiment

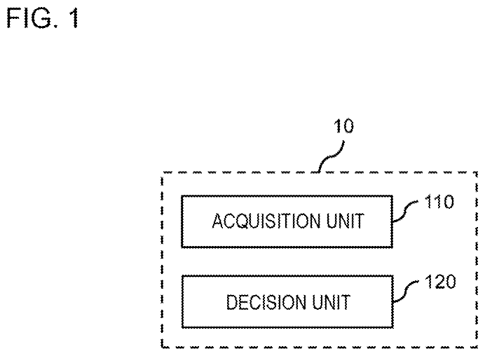

FIG. 1 is a diagram illustrating an overview of a suitability state decision apparatus 10 according to the present example embodiment. The suitability state decision apparatus 10 includes an acquisition unit 110 and a decision unit 120. The acquisition unit 110 acquires a first image being an image when a target takes a medicine. The decision unit 120 generates suitability state information indicating a suitability state of the target relating to the medicine by using the first image.

The target described above is, for example, a person or an animal. In this case, “taking a medicine” is swallowing a medicine, but may be taking a medicine into a body through an injection, a drip, and the like. Note that, the target described above may be a plant. In this case, a medicine may be agricultural chemicals for handling a disease and a pest, or may be fertilizer. Then, “taking a medicine” is spreading chemicals and fertilizer.

The suitability state information includes, for example, at least one of information indicating whether the target feels comfortable to take the medicine and information indicating whether the target feels reluctant to take the medicine. For example, the former information indicates whether a person or an animal feels comfortable to take the medicine orally. Further, for example, the latter information indicates whether a person or an animal feels that the medicine is bitter. The suitability state information may include information indicating a mental reaction when the target takes the medicine.

In this way, the suitability state decision apparatus 10 generates the suitability state information by using the first image. Therefore, the suitability state decision apparatus 10 can support a decision whether the medicine is suitable for the target.

Hereinafter, a detailed example of the suitability state decision apparatus 10 will be described. Hereinafter, a case where the target described above is a person or an animal and the target takes a medicine orally will be described. Thus, in the following description, the “target” will be described as a “target person”.

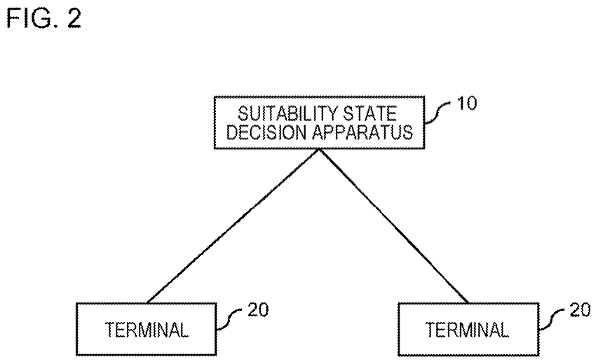

FIG. 2 is diagram illustrating a usage environment of the suitability state decision apparatus 10. The suitability state decision apparatus 10 is used together with a terminal 20. A target person is, for example, a user of a facility such as a nursing home and a hospital or a user of a home care service, which is not limited thereto. The terminal 20 is operated by a person who works at a facility and a person who provides a home care service, for example, by a caregiver and a nurse. Hereinafter, the person will be described as an assistant.

An assistant prepares a medicine that needs to be taken by a target person. The medicine is divided in advance into individual packages each in one dose. Herein, when the target person needs to take a plurality of kinds of medicines at once, the plurality of medicines may be put in one package, or the plurality of medicines may be individually packaged.

An identification marker is provided to a package of a medicine. One example of the identification marker is a bar code and a two-dimensional code. The identification marker includes at least one of information that identifies the medicine put in the package and the amount thereof and information that identifies a target person who needs to take the medicine. Further, the identification marker may be generated for each combination of a target person and a medication timing. Further, the identification marker may include information that identifies a type of the medicine. Herein, the “type of the medicine” indicates that the medicine is any of a tablet, powder (powdered medicine), granules, and a liquid medicine. Note that, the “type of the medicine” may further include presence or absence of a contrivance to make it easy to take the medicine, for example, use or non-use of a wafer or jelly, and may further include information indicating use of either water or hot water when the medicine is taken.

The assistant causes the terminal 20 to generate the first image when the assistant has the target person take the medicine. The first image is generated by capturing an image of the target person when the target person takes the medicine. The first image may be a still image or may be a moving image. Further, the first image may include at least a part of the body of the assistant. In this case, the body of the assistant may indicate, for example, a sign, such as a hand sign, indicating at least one of a start of taking the medicine and an end of taking the medicine.

When the first image is a still image, a capturing timing of the first image includes at least one of a timing immediately after the assistant passes the medicine to the target person, a timing immediately before the target person puts the medicine into a mouth, a time while the target person is swallowing the medicine, and a timing immediately after the target person has swallowed the medicine.

When the first image is a moving image, a capturing start timing of the first image is, for example, a timing immediately before the assistant passes or immediately after the assistant has passed the medicine to the target person, and a capturing end timing of the first image is a timing after the target person has swallowed the medicine. The capturing end timing of the first image may be a timing after the target person has swallowed the medicine and then an image of the inside of a mouth of the target person has been further captured.

Further, when the first image is a moving image or includes a plurality of still images, at least one frame image included in the moving image or at least one of the still images may include an identification marker provided to a package of the medicine.

Then, the assistant transmits the first image to the suitability state decision apparatus 10 by using the terminal 20. The suitability state decision apparatus 10 generates suitability state information by using the first image being transmitted from the terminal 20. Note that, the suitability state decision apparatus 10 can communicate with a plurality of the terminals 20. In other words, one suitability state decision apparatus 10 can handle a plurality of assistants.

Note that, the terminal 20 also transmits information that can identifies the medicine taken by the target person together with the first image to the suitability state decision apparatus Hereinafter, the information will be described as medicine information. The medicine information includes information that identifies at least the medicine, a type thereof, and the amount thereof. One example of the medicine information is, for example, the identification marker described above or information obtained by decoding the identification marker, but may be information being input from the assistant to the terminal 20, such as text information.

Further, the suitability state decision apparatus 10 may generate, by using the first image, information indicating an effect of a medicine taken by the target person in the past, for example, information indicating an effect of a medicine taken last time.

Further, the terminal 20 also transmits, to the suitability state decision apparatus 10, information that can identify the assistant together with the first image or before transmission of the first image. Hereinafter, the information will be described as assistant identification information. The assistant identification information is, for example, biometric information about the assistant, such as a face image, a fingerprint, and an iris. When the assistant identification information is transmitted before the first image, a transmission timing of the assistant identification information is, for example, a timing at which the terminal 20 accesses the suitability state decision apparatus 10. For example, the terminal 20 generates the biometric information about the assistant, and transmits the biometric information to the suitability state decision apparatus 10. Next, the suitability state decision apparatus 10 authenticates the biometric information received from the terminal 20 by using master biometric information stored in advance.

Further, the terminal 20 may generate sound information when the target person takes the medicine described above together with the first image as necessary, and transmit the sound information to the suitability state decision apparatus 10. When the first image is a moving image, the terminal 20 may generate sound information as a part of moving image data.

Further, the terminal 20 may generate a second image being an image of the target person after the target has taken the medicine as necessary. A generation timing of the second image is a timing at which an effect of the medicine being taken is expected to appear, and varies by medicine. The generation timing of the second image is a timing after a predetermined period of time has elapsed since the medicine has been taken, and the predetermined period of time is, for example, equal to or more than an hour and equal to or less than 8 hours. The terminal 20 may transmit, to the suitability state decision apparatus 10, the second image together with the first image or after the first image has been transmitted. In the latter case, the terminal 20 transmits, to the suitability state decision apparatus 10, the second image together with information that can determine the first image associated with the second image.

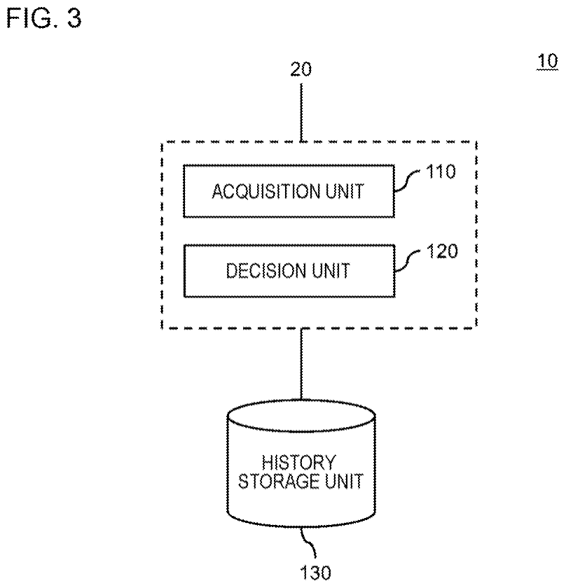

FIG. 3 is a diagram illustrating one example of a functional configuration of the suitability state decision apparatus 10. The suitability state decision apparatus 10 includes a history storage unit 130 in addition to the acquisition unit 110 and the decision unit 120 illustrated in FIG. 1. Note that, the history storage unit 130 may be located outside the suitability state decision apparatus 10.

The acquisition unit 110 acquires a first image transmitted from the terminal 20. At this time, the acquisition unit 110 also acquires medicine information transmitted from the terminal 20. When the terminal 20 transmits sound information to the suitability state decision apparatus 10, the acquisition unit 110 also acquires the sound information. When the terminal transmits a second image to the suitability state decision apparatus 10, the acquisition unit 110 also acquires the second image.

Further, the acquisition unit 110 also acquires assistant identification information transmitted from the terminal 20. Then, the acquisition unit 110 also performs authentication processing on the assistant identification information as necessary, for example, login processing to the suitability state decision apparatus 10.

Then, the acquisition unit 110 stores each piece of the acquired information in the history storage unit 130.

The decision unit 120 generates suitability state information by using the first image. As described by using FIG. 1, the suitability state information indicates a suitability state of a target person relating to a medicine. The decision unit 120 stores, in association with the first image, the suitability state information generated by using the first image in the history storage unit 130. At this time, the decision unit 120 also stores a processing result of the first image in association with the first image in the history storage unit 130. Note that, the decision unit 120 may identify the target person by face authentication. In this case, the processing result of the first image may include identification information about the target person.

A specific example of processing performed by the decision unit 120 is at least one of (1) to (6) below. The decision unit 120 may use at least two of the examples.

(1) The decision unit 120 analyzes at least one of an expression and a feeling when a target person takes a medicine by processing the first image, and generates suitability state information by using the analysis result. For example, when the expression or the feeling is negative, the decision unit 120 lowers a suitability state of the target person relating to the medicine. The “expression or the feeling is negative” means that the target person is reluctant, angry, or sad, which is not limited thereto. On the other hand, when the expression or the feeling is other than that, the decision unit 120 does not lower a suitability state of the target person relating to the medicine. Herein, when the expression or the feeling is positive, the decision unit 120 may increase a suitability state of the target person relating to the medicine. The “expression or the feeling is positive” means that, for example, the target person is happy, which is not limited thereto.

(2) When the first image is a moving image, the decision unit 120 computes a time required until a target person takes a medicine by processing the moving image, and generates suitability state information by using the time. For example, the decision unit 120 lowers a suitability state of the target person relating to the medicine as the time is longer.

Herein, a starting point of the time is a time at which an assistant passes the medicine to the target person, a time at which the target person puts the medicine in a mouth, a time at which the assistant performs a predetermined operation, or a generation date and time of a frame image that succeeds in face authentication. The reason for using the “generation date and time of a frame image that succeeds in face authentication” is that the target person may turn a face away when the target person is reluctant to take the medicine.

Further, an end point of the time is, for example, a time at which the target person opens a mouth after the target person has taken the medicine or a time at which the assistant performs a predetermined operation, which is not limited thereto. Note that, when the first image is a moving image, by processing each of at least two frame images, at least one of an expression and a feeling may be determined for each of the frame images, and a difference between the frame images may be used as suitability state information. In this case, the suitability state information is information relating to the medicine taken by the target person this time. For example, when an expression or a feeling becomes bad after the medicine is taken, the decision unit 120 generates, as the suitability state information, information indicating that the medicine this time is not suitable for the target person. Further, when an expression or a feeling becomes good after the medicine is taken, the decision unit 120 generates, as the suitability state information, information indicating that the medicine this time is suitable for the target person.

(3) When the first image includes an image in which the inside of a mouth of a target person is captured after the target person has swallowed a medicine, the decision unit 120 may decide whether the medicine remains in the mouth of the target person by processing the image, and generate suitability state information by using the decision result. For example, the decision unit 120 lowers a suitability state of the target person relating to the medicine when the medicine remains in the mouth.

(4) The decision unit 120 generates information indicating an effect of medication last time as at least a part of suitability state information by processing the first image acquired this time. For example, when an expression, a movement, and a pose of a target person are good and when disheveled clothing and disheveled hair of the target person are small, the decision unit 120 decides that the medicine taken by the target person last time is effective. On the other hand, when an expression, a movement, and a pose of the target person are bad and when disheveled clothing and disheveled hair of the target person are great, the decision unit 120 decides that the medicine taken last time is not suitable for the target person. Further, when the first image includes an image before a medicine this time is passed to the target person, the suitability state decision apparatus 10 may perform the decision described above by processing this image.

Note that, the decision unit 120 identifies the target person this time. The identification is performed by using a face image included in the first image acquired this time, for example. Then, the decision unit 120 reads information associated with the target person from the history storage unit 130. The information read by the decision unit 120 includes the first image when the same target person takes the same medicine or another medicine in the past, or a processing result of the first image. Then, the decision unit 120 generates the suitability state information by using the information read from the history storage unit 130.

For example, the decision unit 120 may compute the suitability state information by using a comparison result between an expression of the target person in the first image acquired this time, and an expression of the target person in the first image stored in the history storage unit 130, i.e., the first image in the past. For example, when the expression of the target person in the first image this time is better than the expression of the target person in the first image in the past, there is a high possibility that a medicine acquired last time is effective for the target person, and thus information indicating that a medicine taken by the target person this time is suitable for the target person is generated as the suitability state information.

At this time, when the first image includes a face of the target person before the medicine is passed thereto, the decision unit 120 may use a comparison result between an expression of the target person before the medicine this time is passed and an expression of the target person before the medicine last time is passed. When the expression this time is better, it can be inferred that the medicine last time is effective, and thus the decision unit 120 updates the suitability state information about the medicine last time to an increasing direction.

Further, the decision unit 120 first computes temporary suitability state information by performing processing indicated in at least one of (1) to (3) described above on the first image acquired this time. Further, the decision unit 120 reads, from the history storage unit 130, suitability state information about the same target person relating to the same medicine as this time.

Then, when the temporary suitability state information and the read suitability state information indicate the same tendency, the decision unit 120 sets the temporary suitability state information as formal suitability state information. The reason for this is that, when both pieces of the suitability state information indicate a low suitability state, for example, it indicates a high possibility that a cause of the low suitability state is the medicine.

On the other hand, when the temporary suitability state information and the read suitability state information indicate different tendencies, the decision unit 120 corrects the temporary suitability state information in a direction toward the read suitability state information, or adds information indicating that the tendencies of two pieces of the information are different to the suitability state information. The reason for this is that, when the suitability state information this time indicates a low suitability state, there is a possibility that a cause of the low suitability state is caused by other than the medicine.

Further, the decision unit 120 computes the temporary suitability state information, and then reads suitability state information about the same target person relating to a medicine different from the medicine this time. Then, when the temporary suitability state information and the read suitability state information indicate different tendencies, the decision unit 120 sets the temporary suitability state information as formal suitability state information. The reason for this is that it indicates a high possibility that a suitability state this time is caused by the medicine. On the other hand, when both pieces of the temporary suitability state information and the read suitability state information indicate a low suitability state, there is a possibility that a reason for low suitability state information is other than the medicine. Thus, the decision unit 120 sets the temporary suitability state information as formal suitability state information, and then includes, in the suitability state information, information indicating a possibility that a reason for a low suitability state is other than the medicine.

(5) When the terminal 20 transmits sound information to the suitability state decision apparatus 10, the decision unit 120 generates suitability state information by using a result of processing the sound information. The sound information includes at least one of a sound produced by a target person and a sound produced by an assistant.

For example, by converting the sound into text data and processing the text data, the decision unit 120 may recognize each of a word produced by the target person and a word produced by the assistant, and generate the suitability state information by using the recognition result. For example, when the word produced by the target person includes a negative content, the decision unit 120 lowers a suitability state of the target person relating to the medicine. Further, when the word produced by the assistant means a frequent demand for the target person to take the medicine, the decision unit 120 lowers a suitability state of the target person relating to the medicine.

Further, the decision unit 120 may analyze a tone of a sound included in the sound information, and generate the suitability state information by using the analysis result. Further, when a tone of a sound of the target person is high, there is a possibility that the target person is healthy. In this case, there is a high possibility that a medicine acquired last time is effective for the target person, and thus the decision unit 120 increases the suitability state information. Further, when a tone of the assistant is high, there is a high possibility that the assistant repeatedly demands the target person to take the medicine, and thus the decision unit 120 lowers a suitability state of the target person relating to the medicine.

Further, the decision unit 120 may perform a feeling analysis on the sound information, and generate the suitability state information by using the analysis result. A correlation between a feeling and a suitability state is, for example, as described in the example of (1) described above.

(6) When the terminal 20 transmits a second image to the suitability state decision apparatus 10, the decision unit 120 generates at least a part of suitability state information by using the second image. The suitability state information generated herein indicates at least one of whether a medicine is suitable for a physical constitution of a target person, whether the medicine produces an expected effect, and whether a side effect of the medicine occurs. For example, the decision unit 120 analyzes at least one of a physical condition, an expression, and a feeling of the target person by processing the second image, and generates the suitability state information by using the analysis result. For example, when the target person has a negative feeling, or a personal appearance such as clothing and hair of the target person is disheveled, the decision unit 120 decides that a physical condition of the target person is not good, and lowers a suitability state of the medicine. Further, when an expression or a feeling of the target person is negative, the decision unit 120 lowers a suitability state of the target person relating to the medicine.

Then, the decision unit 120 stores, in the history storage unit 130, the generated suitability state information in association with a first image used when the suitability state information is generated.

Note that, when the first image includes code information provided to a package of the medicine, the decision unit 120 may generate medicine information by using the code information.

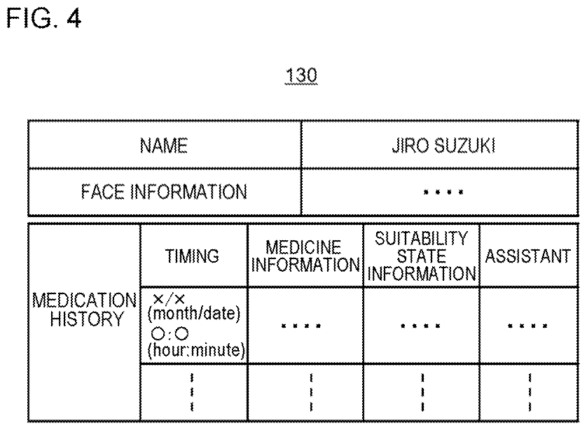

FIG. 4 is a diagram illustrating one example of a data configuration of the history storage unit 130. The history storage unit 130 stores a name, biometric information, and a medication history for each target person. One example of the biometric information is face information, but may be other biometric information such as fingerprint information and vein information. Note that, the biometric information may be stored in a storage unit different from the history storage unit 130. The history storage unit 130 may further store identification information about a target person.

The medication history includes a medication timing and information about a medicine being taken. Herein, one example of the medication timing is a date and time. The information about a medicine being taken may include a kind and the amount of the medicine and a type of the medicine.

Further, when an identification marker is generated for each combination of a target person and a medication timing, the information about a medicine being taken may be an identification marker provided to a package of the medicine or decoded information thereof When the medication history is used, whether the target person takes the medicine as predetermined can be confirmed.

The medication history further includes suitability state information and information that can identify an assistant. The pieces of information are stored for each medication timing. In other words, the pieces of information manage each medication behavior being assisted by which assistant and also manage suitability state information for each medication behavior.

Note that, the medication history may further include at least a part, preferably the whole of a first image. For example, when the medication history includes a face image of the target person, the suitability state decision apparatus 10 can also perform physical condition management of the target person by using the face image. Further, when a contact address of a family of the target person is stored for each target person, the suitability state decision apparatus may transmit, as a situation of the target person, the face image to the contact address of the family. One example of the contact address is an email address or account information of a social media service.

Further, the suitability state decision apparatus 10 may further store information that identifies a person who carries a medicine divided in a package to a facility or a person who packages the medicine, in association with an identification marker of the package of the medicine. One example of the person who carries a medicine to a facility is an employee of a pharmacy, and one example of the person who packages a medicine is a pharmacist. The suitability state decision apparatus 10 may further perform authentication processing on the person who carries the medicine to a facility or the person who packages the medicine when the facility accepts the packaged medicine.

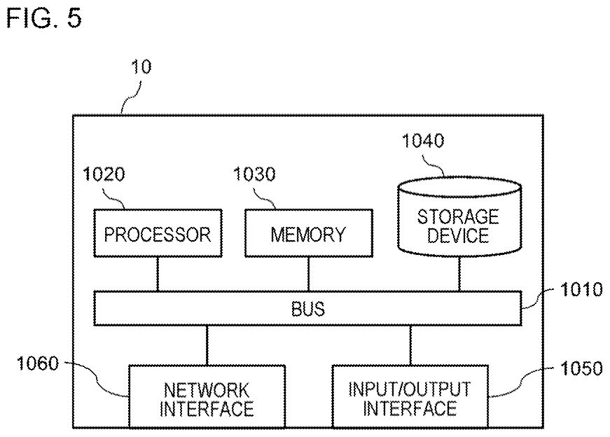

FIG. 5 is a diagram illustrating a hardware configuration example of the suitability state decision apparatus 10. The suitability state decision apparatus 10 includes a bus 1010, a processor 1020, a memory 1030, a storage device 1040, an input/output interface 1050, and a network interface 1060.

The bus 1010 is a data transmission path for allowing the processor 1020, the memory 1030, the storage device 1040, the input/output interface 1050, and the network interface 1060 to transmit and receive data with one another. However, a method for connecting the processor 1020 and the like to one another is not limited to bus connection.

The processor 1020 is a processor achieved by a central processing unit (CPU), a graphics processing unit (GPU), and the like.

The memory 1030 is a main storage achieved by a random access memory (RAM) and the like.

The storage device 1040 is an auxiliary storage achieved by a hard disk drive (HDD), a solid state drive (SSD), a removable medium such as a memory card, a read only memory (ROM), or the like, and includes a storage medium. The storage medium of the storage device 1040 stores a program module that achieves each function (for example, the acquisition unit 110 and the decision unit 120) of the suitability state decision apparatus 10. The processor 1020 reads each program module onto the memory 1030 and executes the program module, and each function associated with the program module is achieved. Further, the storage device 1040 also functions as the history storage unit 130.

The input/output interface 1050 is an interface for connecting the suitability state decision apparatus 10 and various types of input/output equipment.

The network interface 1060 is an interface for connecting the suitability state decision apparatus 10 to a network. The network is, for example, a local area network (LAN) and a wide area network (WAN). A method of connection to the network by the network interface 1060 may be wireless connection or wired connection. The suitability state decision apparatus 10 communicates with the terminal 20 via the input/output interface 1060.

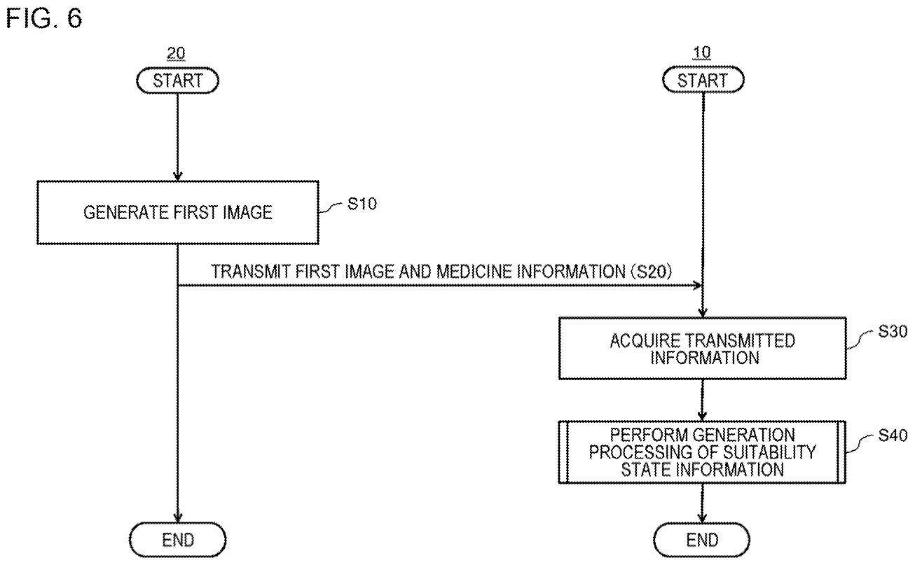

FIG. 6 is a flowchart illustrating one example of processing performed by the suitability state decision apparatus 10 and the terminal 20. Before the processing illustrated in FIG. 6, the suitability state decision apparatus 10 and the terminal 20 perform authentication processing on an assistant in advance. One example of the processing is login processing to the suitability state decision apparatus 10, and is performed as follows, for example. First, the terminal 20 generates biometric information about the assistant, and transmits the biometric information to the suitability state decision apparatus 10. Next, the suitability state decision apparatus 10 authenticates the biometric information received from the terminal 20 by using master biometric information.

Subsequently, the assistant has a target person take a medicine. For example, the assistant takes out a package of the medicine needed to be taken by the target person at a timing, for example, morning, afternoon, or night, and takes out the medicine from the package. Then, the assistant has the target person take the medicine. When the target person takes the medicine, the assistant operates the terminal 20, and generates a first image (step S10). Then, the terminal 20 transmits the first image together with a generation date and time thereof to the suitability state decision apparatus 10. At this time, the terminal 20 also transmits information that can identify the medicine taken by the target person, i.e., medicine information. The medicine information may be code information about the package being included in the first image, or may be information input from the assistant to the terminal 20 (step S20).

The acquisition unit 110 of the suitability state decision apparatus 10 acquires the first image, the generation date and time, and the medicine information (step S30). At this time, since the suitability state decision apparatus 10 and the terminal 20 perform the authentication processing on the assistant in advance, the suitability state decision apparatus 10 can identify the terminal 20, i.e., the assistant being a transmission source of the first image transmitted in step S10. Next, the decision unit 120 of the suitability state decision apparatus 10 performs generation processing of suitability state information by using the first image acquired in step S30 (step S40).

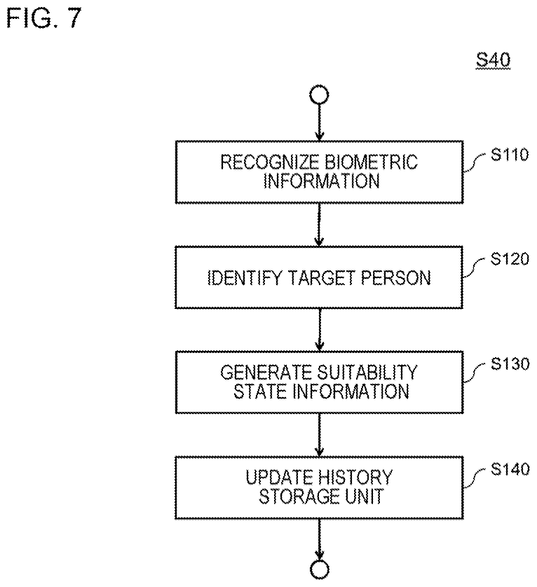

FIG. 7 is a flowchart illustrating a first example of step S40 in FIG. 6. The first image includes face information about the target person. Then, the suitability state decision apparatus 10 identifies the target person by using the face image.

The decision unit 120 recognizes the face information included in the first image (step S110), and identifies the target person who takes the medicine by using the face information (step S120). Herein, when the first image is a moving image in which the target person is captured, the decision unit 120 may select a frame image including a great amount of feature values from among frame images included in the moving image, and generate the face information by processing the frame image.

Then, the decision unit 120 generates suitability state information by using the first image. A specific example of the generation processing of suitability state information is as described by using FIG. 3 (step S130).

Then, the decision unit 120 updates a medication history related to the target person in the history storage unit 130. Specifically, the decision unit 120 stores the generation date and time of the first image as a medication timing, and stores at least the medicine information, the suitability state information, and the information that identifies the assistant in association with the medication timing in the history storage unit 130 (step S140). Herein, the decision unit 120 stores the medicine information in text format in the history storage unit 130. When the terminal 20 transmits code information provided to a package of the medicine as the medicine information, the decision unit 120 converts the code information into information in text format.

Herein, the decision unit 120 may store the first image in the history storage unit 130 as necessary.

Note that, the first image may be generated by using a fixed camera, for example, a monitoring camera instead of the terminal 20. In this case, the suitability state decision apparatus 10 acquires the first image generated by the fixed camera together with the identifier of the fixed camera. Further, the history storage unit 130 stores, for each facility, the identifier of a fixed camera installed in the facility and assistant information about an assistant who works in the facility. Furthermore, the history storage unit 130 stores, for each facility, target person information about a target person who uses the facility. Then, the decision unit 120 determines the facility in which the first image is generated by using the history storage unit 130, and also determines the assistant and the target person being associated with the facility. Processing subsequently performed by the suitability state decision apparatus 10 is as described above.

Further, the first image may include an image in which the inside of a mouth of the target person is captured after the target person has taken a medicine. Hereinafter, the image will be described as a mouth image. Then, the decision unit 120 can determine which target person being associated with the mouth image by processing the face information included in the first image. Note that, when the terminal 20 recognizes the inside of a mouth being present in a capturing range during capturing of a mouth image, the terminal 20 may make illumination brighter. In this way, the terminal 20 can generate a clear mouth image.

Then, the decision unit 120 detects whether the medicine remains in the mouth by processing the face image in a state with the mouth open or the mouth image, and, when the medicine does not remain, the decision unit 120 decides that medication is completed, and updates the medication history. Note that, the processing may not be performed.

Further, the decision unit 120 may store the face image of the state with the mouth open or the mouth image in the history storage unit 130. In this way, it can be confirmed after the event that the medicine has not been left in the mouth of the target person, i.e., that the target person has taken the medicine in the mouth.

When the mouth image is used, the decision unit 120 may set, as a necessary condition for deciding that medication is completed, a condition that a time difference from a generation start of the first image until generation of the mouth image is equal to or less than a reference time. Herein, one example of the reference time is one minute, but may be longer or shorter. Further, the decision unit 120 may identify the target person by using the face image and then request the mouth image from the terminal 20. In this case, a condition that a time from the request for the mouth image until the mouth image is transmitted is equal to or less than a reference time may be set as a necessary condition for deciding that medication is completed. One example of the reference time is also one minute, but may be longer or shorter.

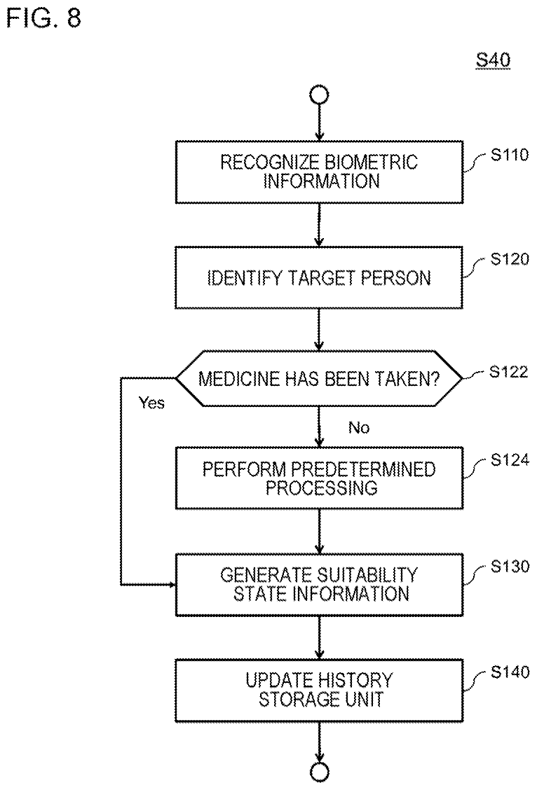

Further, the mouth image may be a moving image. In this case, the decision unit 120 may select, as a processing target, a frame image including a great amount of feature values indicating the inside of the mouth from among frame images constituting the moving image. FIG. 8 is a flowchart illustrating a second example of step S40 in FIG. 6. The example illustrated in FIG. 8 is similar to the first example illustrated in FIG. 7 except for the following points.

The decision unit 120 identifies the target person (step S120), and then decides whether the target person has actually taken the medicine (step S122). For example, as described by using FIG. 7, the decision unit 120 may perform the processing by using a face image in a state with a mouth open or a mouth image. Further, an assistant may input, to the terminal 20, information indicating whether the medicine has been taken, and the terminal 20 may transmit the information to the suitability state decision apparatus 10. In this case, the decision unit 120 decides whether the target person has actually taken the medicine by using the information.

When the target person has not actually taken the medicine (Step S122: No), the decision unit 120 performs predetermined processing (step S124). One example of the predetermined processing is transmitting, to the terminal 20, information to that effect, for example, alert information. When the terminal 20 receives the information, the terminal 20 performs an output, for example, a display indicating that the target person has not actually taken the medicine.

Further, in step S140, the decision unit 120 stores, as a part of medicine information, intake history information indicating whether the target person has actually taken the medicine in the history storage unit 130. Note that, a fact that the target person has not actually taken the medicine refers to a fact that the target person has refused to take the medicine.

Subsequently, when the target person newly takes the same medicine or another medicine and the intake history information about the target person satisfies a predetermined condition, the decision unit 120 outputs information related to the intake history information. An output destination is, for example, the terminal 20.

The predetermined condition is at least one of (a) to (d) below, for example.
(a) Regardless of any kind of a medicine, an event that the medicine has not been taken occurs for a reference number of times or more from a predetermined period ago until the present. The reference number of times may be once or a plurality of times.
(b) Regardless of any kind of a medicine, the medicine has not been taken at the previous medication timing.
(c) An event that the same medicine as a medicine this time has not been taken occurs for a reference number of times or more. This may be counted from a predetermined period ago until the present. The reference number of times may be once or a plurality of times.
(d) The same medicine as a medicine this time has not been taken at the latest medication timing among medication timings at which the medicine is provided.

Further, the output information indicates, for example, a possibility that the target person is reluctant to take the medicine this time. In this way, the assistant can recognize necessity for caution when the medicine this time is taken.

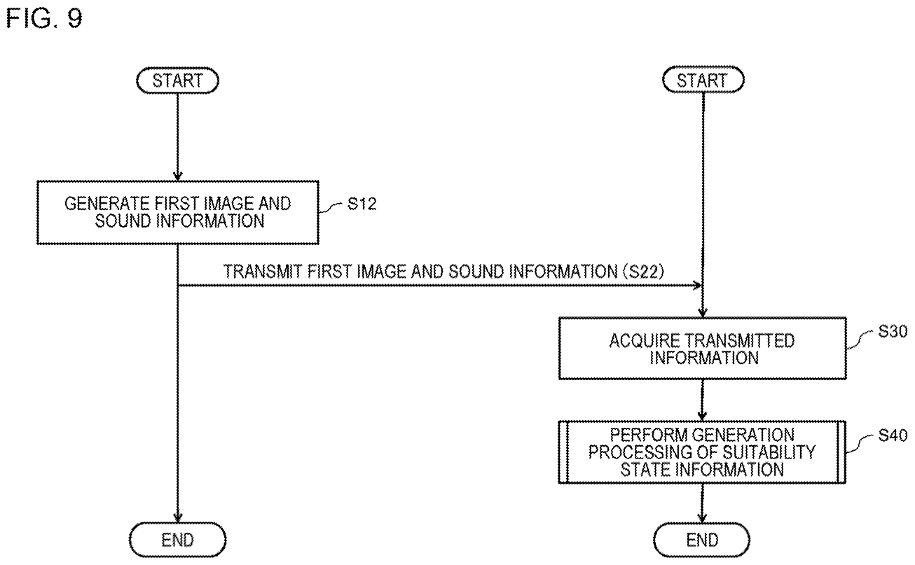

FIG. 9 is a flowchart illustrating a first modification example of FIG. 6. The example illustrated in FIG. 9 is similar to the example illustrated in FIG. 6 except for the following points.

First, the terminal 20 generates sound information in addition to a first image (step S12), and transmits the sound information together with the first image to the suitability state decision apparatus 10 (step S22).

Then, when the decision unit 120 of the suitability state decision apparatus 10 generates suitability state information (step S40), the decision unit 120 uses the sound information in addition to the first image. Details of the processing performed herein are as described by using FIG. 3.

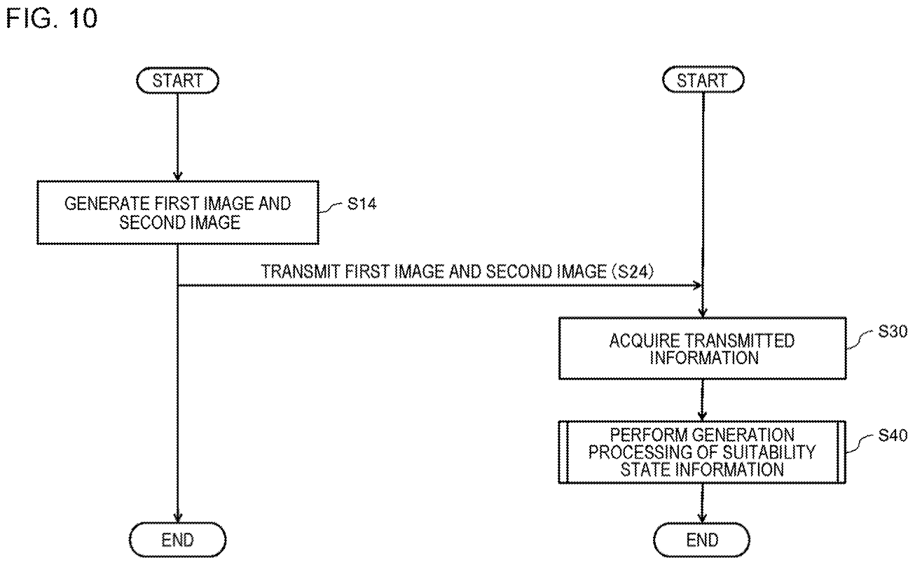

FIG. 10 is a flowchart illustrating a second modification example of FIG. 6. The example illustrated in FIG. 10 is similar to the example illustrated in FIG. 6 except for the following points.

First, the terminal 20 generates a second image in addition to a first image (step S14), and transmits the second image together with the first image to the suitability state decision apparatus 10 (step S24). Note that, when both of the first image and the second image are a moving image, the first image and the second image may be continuous.

Then, when the decision unit 120 of the suitability state decision apparatus 10 generates suitability state information (step S40), the decision unit 120 uses the second image in addition to the first image. Details of the processing performed herein are as described by using FIG. 3.

Note that, in FIG. 10, the sound information illustrated in FIG. 9 may be further used.

Further, the terminal 20 may transmit, to the suitability state decision apparatus 10, the second image at a timing different from that of the first image, for example, a timing after the first image. In this case, the suitability state decision apparatus 10 performs processing using the second image at a timing different from that of processing using the first image.

As described above, according to the present example embodiment, the decision unit 120 of the suitability state decision apparatus 10 generates suitability state information by processing a first image. The suitability state information includes, for example, information indicating whether a target person feels reluctant to take a medicine. Therefore, the suitability state decision apparatus 10 can support a decision whether the medicine is suitable for the target person.

Then, when the suitability state information relating to the medicine is low, an assistant can change a type of the medicine or change the medicine itself to another medicine. By doing so, a possibility that the target person is reluctant to take the medicine is reduced.

Further, according to the present example embodiment, the decision unit 120 generates at least a part of the suitability state information by processing a second image, the second image being an image of the target person after the target has taken the medicine. The suitability state information generated herein indicates at least one of whether the medicine is suitable for a physical constitution of the target person, whether the medicine produces an expected effect, and whether a side effect of the medicine occurs. Thus, the assistant can decide whether it is better to change the kind of the medicine by confirming the suitability state information.

### Second Example Embodiment

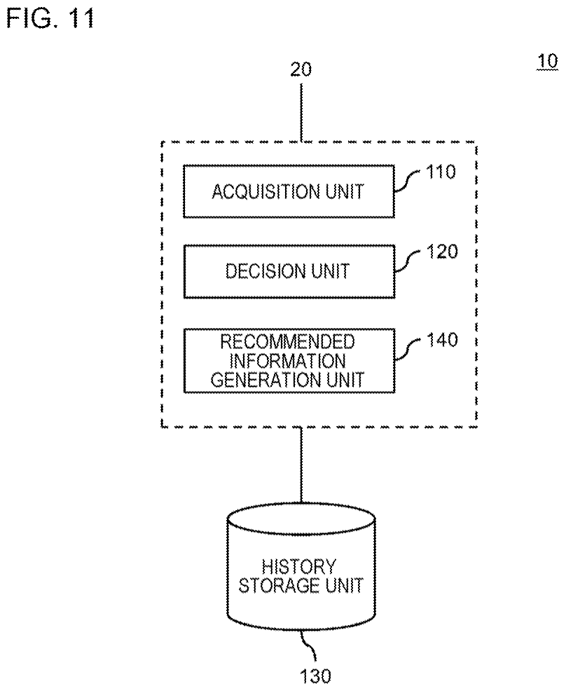

FIG. 11 is a diagram illustrating one example of a functional configuration of a suitability state decision apparatus 10 according to the present example embodiment, and corresponds to FIG. 3 in the first example embodiment. The suitability state decision apparatus according to the present example embodiment is similar to the suitability state decision apparatus 10 according to the first example embodiment except for a point that the suitability state decision apparatus 10 according to the present example embodiment includes a recommended information generation unit 140.

The recommended information generation unit 140 generates, by using suitability state information, information about a medicine that needs to be taken by a target person, and transmits the information to a terminal 20, for example. Hereinafter, the information will be described as recommended information. The recommended information includes, for example, a type of the medicine.

As one example, the recommended information generation unit 140 stores, for each kind of a medicine, a type of the medicine that may be taken by the medicine. As described by using FIG. 2, the “type of the medicine” also includes, in addition to that the medicine is any of a tablet, powder (powdered medicine), granules, and a liquid medicine, presence or absence of a contrivance to make it easy to take the medicine, for example, use or non-use of a wafer or jelly, and information indicating use of either water or hot water. Further, the recommended information generation unit 140 stores information indicating ease of intake for each type of a medicine. Then, when suitability state information is low, the recommended information generation unit 140 first identifies a type of a medicine this time. Then, the recommended information generation unit 140 ascertains whether there is a type easier to be taken than the type this time among types that may be taken by the medicine this time. Then, when there is a type easier to be taken than the type this time, the recommended information generation unit 140 transmits information that recommends the type to the terminal 20.

Further, when there is an interchangeable medicine in terms of effectiveness, the recommended information generation unit 140 may store information indicating the interchangeability. In this case, when suitability state information is low, the recommended information generation unit 140 may determine an interchangeable medicine with the medicine this time, and transmit information indicating the determined medicine as at least a part of recommended information to the terminal 20.

According to the present example embodiment, the suitability state decision apparatus outputs recommended information by using suitability state information. Therefore, a load on an assistant is reduced.

### Third Example Embodiment

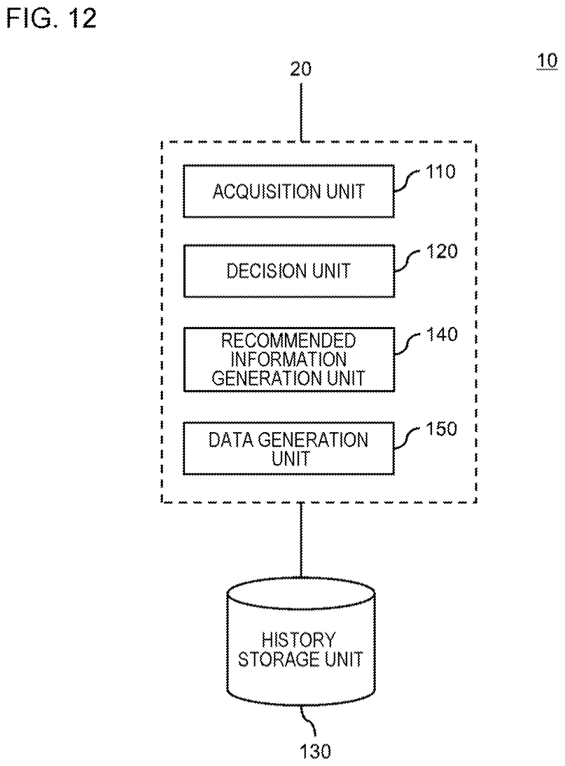

FIG. 12 is a diagram illustrating one example of a functional configuration of a suitability state decision apparatus 10 according to the present example embodiment, and corresponds to FIG. 11 in the second example embodiment. The suitability state decision apparatus 10 according to the present example embodiment is similar to the suitability state decision apparatus 10 according to the second example embodiment except for a point that the suitability state decision apparatus 10 according to the present example embodiment includes a data generation unit 150. Note that, also in the present example embodiment, the suitability state decision apparatus 10 may not include recommended information generation unit 140.

The data generation unit 150 performs statistical processing on information stored in a history storage unit 130.

For example, by processing a history of suitability state information stored in the history storage unit 130 for each target person, the data generation unit 150 may generate at least one of information about a state of the target person and information indicating a tendency of the state.

For example, the data generation unit 150 may infer a state of the target person by performing statistical processing on the suitability state information in a predetermined period (for example, equal to or longer than a week and equal to or shorter than a month). One example of the statistical processing is processing of computing a frequency by state. Herein, the data generation unit 150 may determine a state having a mode. When the statistical processing is performed, a state of the target person can be accurately inferred.

Further, the data generation unit 150 may infer a change in a state of the target person by observing a change in the suitability state information in a predetermined period (for example, equal to or longer than a week and equal to or shorter than a month). The change indicates, for example, whether a state of the target person is better. Further, the data generation unit 150 generates, by target person, correlation information indicating a correlation between the suitability state information and an assistant. One example of the correlation information is a statistical processing result of the suitability state information by target person and by assistant. The statistical processing result is, for example, at least one of an average and a mode, which is not limited thereto. For example, for a certain target person, in a case where the suitability state information when the target person is assisted by a specific assistant is lower than the suitability state information when the target person is assisted by another assistant regardless of a kind of a medicine, it is inferred that the specific assistant and the target person are incompatible with each other. In this case, a manager of the assistant can consider removing the specific assistant from an assistant in charge of the target person.

Note that, for a certain target person, in a case where the suitability state information when the target person is assisted by a specific assistant is lower for a reference number of times or more regardless of a kind of a medicine, the data generation unit 150 may infer that the specific assistant and the target person are incompatible with each other. The reference number of times herein may be once or may be a plurality of times.

Further, the data generation unit 150 may perform statistical processing on the suitability state information by assistant. In the statistical processing, a target person and a kind of a medicine are not considered. The statistical processing result is, for example, at least one of an average and a mode, which is not limited thereto. In the statistical processing, when an average and a mode of a certain assistant is lower than an average and a mode of another assistant, there is a possibility that the assistant has a problem. In this case, a manager of the assistant can consider guiding the assistant.

Note that, the data generation unit 150 may provide at least one of information stored in the history storage unit 130 and a result of performing statistical processing on the information to at least one of a pharmaceutical company, a hospital, a pharmacy, and a facility used by a target person, for example. For example, the data generation unit 150 may perform the statistical processing on the suitability state information by type of a medicine, and provide a result of the statistical processing to a pharmaceutical company. At this time, information subject to the statistical processing is not limited to information about a specific target person, and is information about a plurality of target persons. Further, the data generation unit 150 may perform the statistical processing on the suitability state information by target person and by medicine, and provide a result of the statistical processing to at least one of a hospital, a pharmacy, and a facility used by the target person.

As described above, according to the present example embodiment, the data generation unit 150 of the suitability state decision apparatus 10 generates various types of information by processing suitability state information stored in the history storage unit 130. Therefore, value of the information stored in the history storage unit 130 increases.

### Fourth Example Embodiment

A suitability state decision apparatus 10 according to the present example embodiment is similar to any of the suitability state decision apparatuses 10 according to the first to third example embodiments except for the following points.

First, an acquisition unit 110 acquires information that identifies a target person and a medicine before the target person takes the medicine. Hereinafter, the information will be described as advance information. A specific example of the advance information is similar to a first image, but is different from the first image in a point that the advance information is made by capturing an image before the medicine is taken. For example, the advance information includes at least one of an image in which a package of the medicine is captured and decoded information of an identification marker provided to the package, and biometric information about the target person.

Then, a decision unit 120 sets, as a condition for performing computation processing of suitability state information, a condition that a first image is acquired before a predetermined period of time has elapsed after the advance information has been acquired. When a time since the advance information has been acquired until the first image is acquired is too long, there is a possibility that an assistant performs other work since the assistant has transmitted an advance image until the assistant has the target person take the medicine. In this case, a possibility that the target person accidentally ingests the medicine increases. The present example embodiment allows the assistant to recognize the possibility of the accidental ingestion.

Note that, the acquisition unit 110 of the suitability state decision apparatus 10 may acquire the advance information, and then repeatedly acquire a position of a terminal 20, i.e., a position of the assistant, from the terminal 20. Then, the decision unit 120 may add the position not being changed by a reference value or more to the condition for performing the computation processing of suitability state information. When the position of the terminal 20 is changed by the reference value or more, there is a possibility that the assistant has transmitted the advance information and has then handled another target person. In this case, there is a possibility that the assistant makes a mistake in a medicine or makes a mistake in a target person who needs to take a medicine.

Note that, the terminal 20 instead of the suitability state decision apparatus 10 may confirm that the position of the terminal 20 is not changed by the reference value or more.

While the example embodiments of the present invention have been described with reference to the drawings, the example embodiments are only exemplification of the present invention, and various configurations other than the above-described example embodiments can also be employed.

Further, the plurality of steps (pieces of processing) are described in order in the plurality of flowcharts used in the above-described description, but an execution order of steps performed in each of the example embodiments is not limited to the described order. In each of the example embodiments, an order of illustrated steps may be changed within an extent that there is no harm in context. Further, each of the example embodiments described above can be combined within an extent that a content is not inconsistent.

A part or the whole of the above-described example embodiment may also be described in supplementary notes below, which is not limited thereto.

1. A suitability state decision apparatus including:
an acquisition unit that acquires a first image being an image when a target takes a medicine; and
a decision unit that generates, by using the first image, suitability state information indicating a suitability state of the target relating to the medicine.

2. The suitability state decision apparatus according to supplementary note 1 described above, in which
the target is a person or an animal, and
the decision unit analyzes, by processing the first image, at least one of an expression and a feeling when the target takes the medicine, and generates the suitability state information by using a result of the analysis.

3. The suitability state decision apparatus according to supplementary note 1 or 2 described above, in which
the target is a person or an animal, and
the decision unit generates, as the suitability state information, information indicating an effect of a medicine taken by the target in the past.

4. The suitability state decision apparatus according to any one of supplementary notes 1 to 3 described above, in which
the target is a person or an animal,
the first image is a moving image, and
the decision unit computes, by processing the moving image, a time required until the target takes the medicine, and generates the suitability state information by using the time.

5. The suitability state decision apparatus according to any one of supplementary notes 1 to 4 described above, in which
the acquisition unit acquires a second image being an image of the target after the target has taken the medicine, and
the decision unit further generates at least a part of the suitability state information by using the second image.

6. The suitability state decision apparatus according to any one of supplementary notes 1 to 5 described above, in which
the target is a person or an animal,
the medicine is orally administered, and
the first image includes an image in which an inside of a mouth of the target is captured after the target has taken the medicine.

7. The suitability state decision apparatus according to any one of supplementary notes 1 to 6 described above, in which
the decision unit further generates the suitability state information by using the first image when the target takes the medicine or another medicine in the past, or a processing result of the first image.

8. The suitability state decision apparatus according to any one of supplementary notes 1 to 7 described above, in which
the target is a person or an animal,
the acquisition unit further acquires sound information generated when the target takes the medicine, and
the decision unit further generates the suitability state information by using the sound information.

9. The suitability state decision apparatus according to any one of supplementary notes 1 to 8 described above, further including
a recommended information generation unit that generates, by using the suitability state information, recommended information being information about a medicine that needs to be taken by the target.

10. The suitability state decision apparatus according to supplementary note 9 described above, in which
the recommended information includes a type of the medicine.

11. The suitability state decision apparatus according to any one of supplementary notes 1 to 10 described above, in which
an assistant accompanies the target when the target takes the medicine, and
the decision unit stores the suitability state information in association with a combination of the target and the assistant when the target takes the medicine in a storage unit,
the suitability state decision apparatus further including a data processing unit that generates, by the target, correlation information indicating a correlation between the suitability state information and the assistant by using information stored in the storage unit.

12. The suitability state decision apparatus according to any one of supplementary notes 1 to 11 described above, in which
an assistant accompanies the target when the target takes the medicine, and
the decision unit stores the suitability state information in association with the assistant when the target takes the medicine in a storage unit,
the suitability state decision apparatus further including a data processing unit that generates, by the assistant, statistical information about the suitability state information by using information stored in the storage unit.

13. The suitability state decision apparatus according to any one of supplementary notes 1 to 12 described above, in which
the target is a person or an animal, and
the decision unit further decides whether the target has taken the medicine, and performs predetermined processing when the target has not taken the medicine.

14. The suitability state decision apparatus according to supplementary note 13 described above, in which
the decision unit
   stores intake history information indicating whether the target has taken the medicine in a storage unit, and
   outputs information about the intake history information when the target newly takes the medicine or another medicine and the intake history information about the target satisfies a predetermined condition.

15. A suitability state decision method including,
by a computer:
acquiring a first image being an image when a target takes a medicine; and
generating, by using the first image, suitability state information indicating a suitability state of the target relating to the medicine.

16. The suitability state decision method according to supplementary note 15 described above, in which
the target is a person or an animal,
the suitability state decision method further including, by the computer, analyzing, by processing the first image, at least one of an expression and a feeling when the target takes the medicine, and generating the suitability state information by using a result of the analysis.

17. The suitability state decision method according to supplementary note 15 or 16 described above, in which
the target is a person or an animal,
the suitability state decision method further including, by the computer, generating, as the suitability state information, information indicating an effect of a medicine taken by the target in the past.

18. The suitability state decision method according to any one of supplementary notes 15 to 17 described above, in which
the target is a person or an animal, and
the first image is a moving image,
the suitability state decision method further including, by the computer, computing, by processing the moving image, a time required until the target takes the medicine, and generating the suitability state information by using the time.

19. The suitability state decision method according to any one of supplementary notes 15 to 18 described above, further including,
by the computer:
acquiring a second image being an image of the target after the target has taken the medicine; and
further generating at least a part of the suitability state information by using the second image.

The suitability state decision method according to any one of supplementary notes 15 to 19 described above, in which
the target is a person or an animal,
the medicine is orally administered, and
the first image includes an image in which an inside of a mouth of the target is captured after the target has taken the medicine.

21. The suitability state decision method according to any one of supplementary notes 15 to 20 described above, further including,
by the computer,
further generating the suitability state information by using the first image when the target takes the medicine or another medicine in the past, or a processing result of the first image.

22. The suitability state decision method according to any one of supplementary notes 15 to 21 described above, in which
the target is a person or an animal,
the suitability state decision method further including,
by the computer:
further acquiring sound information generated when the target takes the medicine; and
further generating the suitability state information by using the sound information.

23. The suitability state decision method according to any one of supplementary notes 15 to 22 described above, further including,
by the computer,
generating, by using the suitability state information, recommended information being information about a medicine that needs to be taken by the target.

24. The suitability state decision method according to supplementary note 23 described above, in which
the recommended information includes a type of the medicine.

25. The suitability state decision method according to any one of supplementary notes 15 to 24 described above, in which
an assistant accompanies the target when the target takes the medicine,
the suitability state decision method further including,
by the computer:
storing the suitability state information in association with a combination of the target and the assistant when the target takes the medicine in a storage unit; and
generating, by the target, correlation information indicating a correlation between the suitability state information and the assistant by using information stored in the storage unit.

26. The suitability state decision method according to any one of supplementary notes 15 to 25 described above, in which
an assistant accompanies the target when the target takes the medicine,
the suitability state decision method further including,
by the computer:
storing the suitability state information in association with the assistant when the target takes the medicine in a storage unit; and
generating, by the assistant, statistical information about the suitability state information by using information stored in the storage unit.

27. The suitability state decision method according to any one of supplementary notes 15 to 26 described above, in which
the target is a person or an animal,
the suitability state decision method further including, by the computer, further deciding whether the target has taken the medicine, and performing predetermined processing when the target has not taken the medicine.

28. The suitability state decision method according to supplementary note 27 described above, further including,
by the computer:
storing intake history information indicating whether the target has taken the medicine in a storage unit; and
outputting information about the intake history information when the target newly takes the medicine or another medicine and the intake history information about the target satisfies a predetermined condition.

29. A program causing a computer to:
acquire a first image being an image when a target takes a medicine; and
generate, by using the first image, suitability state information indicating a suitability state of the target relating to the medicine.

30. The program according to supplementary note 29 described above, in which
the target is a person or an animal,
the program further causing the computer to analyze, by processing the first image, at least one of an expression and a feeling when the target takes the medicine, and generate the suitability state information by using a result of the analysis.

31. The program according to supplementary note 29 or 30 described above, in which
the target is a person or an animal,
the program further causing the computer to generate, as the suitability state information, information indicating an effect of a medicine taken by the target in the past.

32. The program according to any one of supplementary notes 29 to 31 described above, in which
the target is a person or an animal, and
the first image is a moving image,
the program further causing the computer to compute, by processing the moving image, a time required until the target takes the medicine, and generate the suitability state information by using the time.

33. The program according to any one of supplementary notes 29 to 32 described above, further causing the computer to:
acquire a second image being an image of the target after the target has taken the medicine; and
further generate at least a part of the suitability state information by using the second image.

34. The program according to any one of supplementary notes 29 to 33 described above, in which
the target is a person or an animal,
the medicine is orally administered, and
the first image includes an image in which an inside of a mouth of the target is captured after the target has taken the medicine.

35. The program according to any one of supplementary notes 29 to 34 described above, further causing the computer to
further generate the suitability state information by using the first image when the target takes the medicine or another medicine in the past, or a processing result of the first image.

36. The program according to any one of supplementary notes 29 to 35 described above, in which
the target is a person or an animal,
the program further causing the computer to:
further acquire sound information generated when the target takes the medicine; and
further generate the suitability state information by using the sound information.

37. The program according to any one of supplementary notes 29 to 36 described above, further causing the computer to
generate, by using the suitability state information, recommended information being information about a medicine that needs to be taken by the target.

38. The program according to supplementary note 37 described above, in which
the recommended information includes a type of the medicine.

39. The program according to any one of supplementary notes 29 to 38 described above, in which
an assistant accompanies the target when the target takes the medicine,
the program further causing the computer to:
perform processing of storing the suitability state information in association with a combination of the target and the assistant when the target takes the medicine in a storage unit; and
generate, by the target, correlation information indicating a correlation between the suitability state information and the assistant by using information stored in the storage unit.

40. The program according to any one of supplementary notes 29 to 39 described above, in which
an assistant accompanies the target when the target takes the medicine,
the program further causing the computer to:
perform processing of storing the suitability state information in association with the assistant when the target takes the medicine in a storage unit; and
generate, by the assistant, statistical information about the suitability state information by using information stored in the storage unit.

41. The program according to any one of supplementary notes 29 to 40 described above, in which
the target is a person or an animal,
the program further causing the computer to further decide whether the target has taken the medicine, and perform predetermined processing when the target has not taken the medicine.

42. The program according to supplementary note 41 described above, further causing the computer to:
perform processing of storing intake history information indicating whether the target has taken the medicine in a storage unit; and
output information about the intake history information when the target newly takes the medicine or another medicine and the intake history information about the target satisfies a predetermined condition.

43. A computer-readable storage medium storing the program according to any one of supplementary notes 29 to 42 described above.

It is apparent that the present invention is not limited to the above embodiment, and may be modified and changed without departing from the scope and spirit of the invention.

### REFERENCE SIGNS LIST

10 Suitability state decision apparatus
20 Terminal
110 Acquisition unit
120 Decision unit
130 History storage unit
140 Recommended information generation unit
150 Data generation unit

What is claimed is:

1. A suitability state decision apparatus comprising:
at least one memory configured to store instructions; and
at least one processor configured to execute the instructions to perform operations, the operations comprising:
acquiring a first image being an image when a target takes a medicine; and
generating, by using the first image, suitability state information indicating a suitability state of the target relating to the medicine.

2. The suitability state decision apparatus according to claim 1, wherein
the target is a person or an animal, and
the operations comprise analyzing, by processing the first image, at least one of an expression and a feeling when the target takes the medicine, and generating the suitability state information by using a result of the analysis.

3. The suitability state decision apparatus according to claim 1, wherein
the target is a person or an animal, and
the operations comprise generating, as the suitability state information, information indicating an effect of a medicine taken by the target in a past.

4. The suitability state decision apparatus according to claim 1, wherein
the target is a person or an animal,
the first image is a moving image, and
the operations comprise computing, by processing the moving image, a time required until the target takes the medicine, and generating the suitability state information by using the time.

5. The suitability state decision apparatus according to claim 1, wherein the operations comprise
acquiring a second image being an image of the target after the target has taken the medicine, and
generating at least a part of the suitability state information by using the second image.

6. The suitability state decision apparatus according to claim 1, wherein the operations comprise
generating the suitability state information by using the first image when the target takes the medicine or another medicine in a past, or a processing result of the first image.

7. The suitability state decision apparatus according to claim 1, wherein
the target is a person or an animal,
the operations comprise
acquiring sound information generated when the target takes the medicine, and
generating the suitability state information by using the sound information.

8. The suitability state decision apparatus according to claim 1, wherein
the target is a person or an animal, and
the operations comprise deciding whether the target has taken the medicine, and performing predetermined processing when the target has not taken the medicine.

9. A suitability state decision method comprising,
by a computer:
acquiring a first image being an image when a target takes a medicine; and
generating, by using the first image, suitability state information indicating a suitability state of the target relating to the medicine.

10. The suitability state decision method according to claim 9, wherein
the target is a person or an animal, and
the method further comprises analyzing, by processing the first image, at least one of an expression and a feeling when the target takes the medicine, and generating the suitability state information by using a result of the analysis.

11. The suitability state decision method according to claim 9, wherein
the target is a person or an animal, and
the method further comprises generating, as the suitability state information, information indicating an effect of a medicine taken by the target in a past.

12. The suitability state decision method according to claim 9, wherein
the target is a person or an animal,
the first image is a moving image, and
the method further comprises computing, by processing the moving image, a time required until the target takes the medicine, and generating the suitability state information by using the time.

13. The suitability state decision method according to claim 9, wherein the method further comprises
acquiring a second image being an image of the target after the target has taken the medicine, and
generating at least a part of the suitability state information by using the second image.

14. The suitability state decision method according to claim 9, wherein the method further comprises
generating the suitability state information by using the first image when the target takes the medicine or another medicine in a past, or a processing result of the first image.

15. A non-transitory computer-readable medium storing a program for causing a computer to perform operations, the operations comprising:
acquiring a first image being an image when a target takes a medicine; and
generating, by using the first image, suitability state information indicating a suitability state of the target relating to the medicine.

16. The non-transitory computer-readable medium according to claim 15, wherein
the target is a person or an animal, and
the operations comprise analyzing, by processing the first image, at least one of an expression and a feeling when the target takes the medicine, and generating the suitability state information by using a result of the analysis.

17. The non-transitory computer-readable medium according to claim 15, wherein
the target is a person or an animal, and
the operations comprise generating, as the suitability state information, information indicating an effect of a medicine taken by the target in a past.

18. The non-transitory computer-readable medium according to claim 15, wherein
the target is a person or an animal,
the first image is a moving image, and
the operations comprise computing, by processing the moving image, a time required until the target takes the medicine, and generating the suitability state information by using the time.

19. The non-transitory computer-readable medium according to claim 15, wherein the operations comprise
acquiring a second image being an image of the target after the target has taken the medicine, and
generating at least a part of the suitability state information by using the second image.

20. The non-transitory computer-readable medium according to claim 15, wherein the operations comprise
generating the suitability state information by using the first image when the target takes the medicine or another medicine in a past, or a processing result of the first image.
